(19) United States
(12) Patent Application Publication
(10) Pub. No.: US 2003/0091612 A1
(43) Pub. Date: May 15, 2003

(54) Antimicrobial polyolefin articles and methods for their preparation

(76) Inventor: Subramaniam Sabesan, Wilmington, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/288,762

(22) Filed: Nov. 6, 2002

Related U.S. Application Data
(60) Provisional application No. 60/338,529, filed on Nov. 6, 2001.

(51) Int. Cl. A61K 9/70; C08K 3/00

(52) U.S. Cl. 424/423; 523/122; 424/443

(57) ABSTRACT

This invention relates to antimicrobial polyolefin articles utilizing chitosan and chitosan-metal complexes as the antimicrobial agent and methods for making same.

### FIELD OF THE INVENTION

This invention relates to antimicrobial polyolefin articles utilizing chitosan and chitosan-metal complexes as the antimicrobial agent and methods for making same. TECHNICAL BACKGROUND OF THE INVENTION

This invention relates to the use of chitosan and chitosan-metal complexes to generate polyolefin articles having antimicrobial properties.

As evidenced by the presence in the market of numerous materials for eliminating or minimizing human contact with bacteria, there is clearly a demand for materials and/or processes that either minimize or kill bacteria encountered in the environment. Such materials are useful in areas of food preparation or handling and in areas of personal hygiene, such as bathrooms. Similarly, there is a use for such antibacterial materials in hospitals and nursing homes where people with lowered resistance are especially vulnerable to bacteria.

Chitosan is the commonly used name for poly-[1-4]-β-D-glucosamine. Chitosan is chemically derived from chitin which is a poly-[1-4]-β-N-acetyl-D-glucosamine, which, in turn, is derived from the cell walls of fungi, the shells of insects and, especially, crustaceans. Thus, it is inexpensively derived from widely available materials. It is available as an article of commerce from, for example, Biopolymer Engineering, Inc. (St. Paul, Minn.); Biopolymer Technologies, Inc. (Westborough, Mass.); and CarboMer, Inc. (Westborough, Mass.).

Chitosan can be treated with metal salt solutions so that the metal ion forms a complex with the chitosan. Chitosan and chitosan-metal compounds are known to provide antimicrobial activity (see, e.g., T. L. Vigo, “Antimicrobial Polymers and Fibers: Retrospective and Prospective,” in Bioactive Fibers and Polymers, J. V. Edwards and T. L. Vigo, eds., ACS Symposium Series 792, pp. 175-200, American Chemical Society, 2001).

In U.S. Patent Application No. 60/290,297, chitosan is shown to impart antimicrobial activity to polyester articles when applied in the form of an acidic solution. The article may be treated subsequently with a solution of zinc sulfate, cupric sulfate, or silver nitrate to enhance antimicrobial activity.

PCT application WO 00/49219 discloses the preparation of substrates with biocidal properties. The deposition of solubilized chitosan on polypropylene, among other materials, followed by treatment with silver salts, reduction of the silver salt and crosslinking the chitosan is disclosed to yield a durable biocidal article. Substrates are fibrous articles. Further, the application of silver salts is followed by a chemical reduction step. The disclosure also requires the crosslinking of the chitosan after it is applied and either before or after the silver salt treatment, which is also not required by the present invention.

Rasmussen et al. (J. Am. Chem. Soc. 99 (14), 4736-45, 1977) oxidized low density polyethylene film with concentrated chromic acid, followed by oxidation with 70% HNO₃. This generated a surface containing a small number of different types of functional groups. The surface functionality consisted mainly of carbonyl derivatives, with approximately 60% of these present as carboxylic acid groups and 40% as ketones or aldehydes. This allows further reactions on the polymer surface.

U.S. Pat. No. 4,326,532 discloses preparation of polymeric surfaces for bonding with chitosan by three methods: (1) with oxygen R_{f} plasma discharge; (2) chromic acid oxidation; or (3) R_{f} plasma polymerization of acids on the surface. The first method exemplified. Chitosan-coated polyethylene articles were prepared as controls for chitosan-coated polyethylene articles onto which a layer of heparin was bonded to provide antithrombogenic articles that could be useful as implants. In a paper co-authored by the inventor (L. K. Lambrecht et al., Trans. Am. Soc. Artif. Intern. Organs, Vol. XXVII, 380-385, 1981), on transient thrombus deposition on chitosan-heparin coated polyethylene, polyethylene tubings are primed for chitosan coating by exposure to a chromic acid solution. In both of these references, the chitosan/polyethylene articles are only experimental controls and are not under consideration as useful articles in their own right.

U.S. Pat. No. 6,042,877 discloses a method for making antimicrobial articles by coating a solution of chitosan and a metal ion onto a substrate and adding a potentiator, such as an alkyl dithiocarbamate. Substrates include, for example, poly(vinyl chloride) sheeting, fibrous substrates (including polyolefin fibers), and nonwoven webs. Articles of interest are intended for cleaning, scrubbing or wiping, such as brushes, sponges, mops, towels, and bibs. Japanese Kokai 05269181 discloses the preparation of antimicrobial polymers for contact lenses and containers for contact lenses. The reference discusses chitosan being reacted with the surface of an optically clear contact lens material. Exemplified are methacrylate/carbonate copolymers with hydroxyl functionality. In one example, chitosan is attached to the surface by graft polymerization in carbodiimide aqueous solution onto an acrylic acid layer that has been first grafted onto the contact lens. In another example, a solution of chitosan in N-methyl-pyrrolidone contacts the contact lens, and the chitosan is crosslinked.

U.S. Pat. No. 5,618,622 discloses a surface-modified fibrous filtration medium which includes hydrocarbon polymer fibers having cationic or anionic functional groups on the surfaces thereof, coated with a polyelectrolyre of opposite charge, such as chitosan. There is no mention of antimicrobial properties.

U.S. Pat. No. 6,197,322 discloses an antimicrobial structure comprising coating a hydrophobic surface of a solid substrate, such as a polypropylene nonwoven fabric, with a chitosan material. Such coated fabric can be used as the body side liner in a personal care garment to reduce odor and promote skin wellness. The chitosan does not react chemically with the hydrophobic surface. A crosslinking agent can be used to insolubilize the chitosan coating on the surface.

It is an object of this invention to provide antimicrobial polyolefin articles in which the antimicrobial element comprises chitosan. Also provided are methods for the production of such polyolefin articles.

### SUMMARY OF THE INVENTION

The invention discloses an antimicrobial polyolefin article having chitosan grafted thereon. And

2. The antimicrobial polyolefin article of claim 1 further comprising one or more compounds selected from the group consisting of metal salts, carboxyl-containing polymers, and combinations thereof.

### DETAILED DESCRIPTION OF THE INVENTION

The present invention is directed to antimicrobial polyolefin articles. By “polyolefin article” is meant an article whose surface is at least 50% by area a polyolefin homopolymer or polyolefin copolymer. Articles prepared by the methods of the invention exhibit antimicrobial functionality wherein microbial growth is reduced as the article is commonly used. The term “antimicrobial” as used herein, means both bactericidal and fungicidal as is commonly known in the art. By “antimicrobial growth is reduced” or “reduction of bacterial growth” is meant that a 99.9% kill of the bacteria in 24 hours has been met as measured by the Shake Flask test described below and as is commonly used to measure antimicrobial functionality which indicates a minimum requirement of a 3-log reduction in bacterial growth.

The articles of the present invention have at least one layer of chitosan grafted thereon. Chitosan is the commonly used name for poly-[1-4]-β-D-glucosamine. Chitosan is chemically derived from chitin which is a poly-[1-4]-β-N-acetyl-D-glucosamine which, in turn, is derived from the cell walls of fungi, the shells of insects and, especially, crustaceans.

As used herein, the term “grafted” means that the chitosan is bound to the polyolefin substrate by either ionic (electrostatic) or covalent bonding. Grafting of the chitosan to the polyolefin article may be confirmed by Electron Spectroscopy for Chemical Analysis (ESCA) [see, for example, Xin Ou, Anders Wirsen, Bjorn Orlander, Anne-Christine Albertsson, Polymer Bulletin, (2001), vol. 46., pp.223-229 and Huh, M. W., Kang, I., Lee, D. H., Kim, W. S., Lee, D. H., Park, L. S., Mln, K. E., and Seo, K. H., J. Appl. Polym. Sci. (2001), vol. 81, p. 2769]. Grafting is also established by the literature report of Ga-er Yu, Frederick G. Morin, Geffory A. R. Nobes, and Robert H. Marchessault, in Macromolecules, (1999), vol. 32, pp. 518-520). ESCA data demonstrate that the chitosan-modified surfaces of the polyolefin articles of the present invention are similar in composition to those of the chitosan starting materials. The ESCA data also show that these surfaces have a significant level of nitrogen that is incorporated in a salt form, which provides evidence that the chitosan in physically linked to the surface through ionic interactions.

Polymers suitable as the substrate component of the present invention are olefinic homopolymers such as polypropylene, polyethylenes such as low density polyethylene, linear low density polyethylene, high density polyethylene, ultra low density polyethylene, metallocene polyethylene, high density polyethylene and ultra high molecular weight polyethylene, copolymers of ethylene and vinyl esters such as vinyl acetate, and copolymers of ethylene and unsaturated acid or esters of those acids such as acrylic or methacrylic acid, or 1-8 carbon alkyl acrylates and methacrylates, or mixtures of these comonomers. Also included are ionomers of ethylene/acrylic acid or methacrylic acid copolymers and terpolymers. Ionomers are the well known metal ion partially neutralized ethylene/(meth)acrylic acid copolymers, described in U.S. Pat. No. 3,264,272 (Rees) which is hereby incorporated by reference. The preferred polyolefins useful herein are polyethylene and copolymers and blends thereof.

As an optional first step of the present invention, the outer surface of the polyolefin article is cleaned. The surface of the polyolefin article can be cleaned with C₁ to C₆ alcohols, dialkyl formamide and acetamide or with other polar solvents capable of extracting plasticizers. In a preferred embodiment, the polyolefin surface is cleaned with hot alcohol (about 70 to about 80° C.) for about 15 to about 24 hours. The surface of the article may then be dried by methods commonly known in the art, for example, by vacuum, ambient air drying, oven drying, and air forced drying.

After cleaning and drying the surface, the polyolefin articles are then pretreated. During pretreatment, the polyolefin articles are acidified in order to prepare their surface for subsequent attachment of chitosan groups. The pretreatment of the present invention involves oxidizing the polyolefin with chromic acid according to the procedure described in Rasmussen et al. cited supra.

The pretreatment step comprises exposure of the article to a concentrated aqueous solution of chromic oxide (Cr₂O₃) and sulfuric acid; washing with deionized water; exposure to concentrated acid (70% nitric acid or 6N hydrochloric acid) to remove chromic salt residues; and further, thorough washing with deionized water. Specifics of the pretreatment step will depend on plasticizers and other additives present in the particular sample. The temperature of the chromic acid solution will affect the rate of surface oxidation, as shown in Rasmussen, FIG. 8. Typical temperatures for the process are from ambient to about 80° C. for the chromic acid/sulfuric acid mixture, more typically from about 65 to about 80° C. The ratio by weight of chromic oxide:water:sulfuric acid can be about 25-30: 40-50:25-30. The ratio 29:42:29 is most preferred for producing a high density of carbonyl groups at the surface. The nitric or hydrochloric acid temperature is typically from about 40° C. to about 60° C. The water wash temperature maybe from ambient to about 70° C.

Following the acidification pretreatment step, the article is treated with chitosan under grafting conditions. This comprises soaking or wetting the article with a chitosan treating solution. Typically, this treating solution is an aqueous acetic acid solution, preferably about 0.5% to about 5% aqueous acetic acid. In a preferred embodiment, an aqueous solution containing 1% to 2% chitosan and 0.5% to 1.0% acetic acid is prepared. In more a preferred embodiment, an aqueous solution containing 2% chitosan and 0.75% acetic acid is prepared. In another preferred embodiment, 2% chitosan and 1.5% aqueous acetic acid solution is prepared. The time of treatment is typically 5 to 30 minutes. The temperature of the treatment is not critical; room temperature is preferred.

After treatment with chitosan under grafting conditions, the article may be washed, preferably with deionized water. Optionally, the article may then be dried via methods known in the art. Such methods include, ambient air drying, oven drying, and air forced drying. In a preferred embodiment, the polyolefin articles are oven dried at about 70-90° C., more preferably at about 80° C., for about 12 to about 24 hours.

In a preferred embodiment of the method of the present invention, the polyolefin article is cleaned by Soxhlet extraction with hot 2-propanol, then dried under vacuum. The article is then treated with a solution of chromium (VI) oxide-water-sulfuric acid (29:42:29 wt. ratio) for 5 to 10 min at 72° C., washed three times with deionized water, then soaked in concentrated nitric acid at 50° C. for 15 min. The article is then extensively washed with deionized water to remove the bulk of the mineral acid.

Articles prepared by the methods of the present invention exhibit antibacterial properties. Said antibacterial properties may, optionally, be further enhanced by treatment with metal salts. Metal salts useful for the present invention include, for example, zinc sulfate, copper sulfate, silver nitrate, soluble zinc, copper, and silver salts. The metal salts are typically applied by dipping, spraying or padding a dilute (0.1% to 5%) solution of the salt in water onto the article.

The preferred articles of the present invention provide multiple uses. The following are examples of applications wherein microbial growth is reduced in the end-use for which the particular application is commonly used.

The articles of the invention include packaging for food, personal care (health and hygiene) items, and cosmetics. By “packaging” is meant either an entire package or a component of a package. Examples of packaging components include but are not limited to packaging film, liners, caps, and lids. The package may be in any form appropriate for the particular application, such as a can, box, bottle, jar, bag, or closed-ended tube. The packaging may be fashioned by any means known in the art, such as by extrusion, coextrusion, thermoforming, injection molding, lamination, or blow molding.

Some specific examples of packaging include, but are not limited to bottles, tips, applicators, and caps for prescription and non-prescription capsules and pills; solutions, creams, lotions, powders, shampoos, conditioners, deodorants, antiperspirants, and suspensions for eye, ear, nose, throat, vaginal, urinary tract, rectal, skin, and hair contact; lip product packaging, and caps. Examples of applicators included lipstick, chapstick, and gloss; packages and applicators for eye cosmetics, such as mascara, eyeliner, shadow, dusting powder, bath powder, blusher, foundation and creams. These applicators are used to apply substances onto the various surfaces of the body and reduction of bacterial growth will be beneficial in such applications. Other forms of packaging include drink bottle necks, replaceable caps, non-replaceable caps, and dispensing systems; food and beverage delivery systems; baby bottle nipples and caps; and pacifiers. Wherein a liquid, solution or suspension is intended to be applied, the package may be fashioned for application in a form for dispensing discrete drops or for spraying of droplets. The invention will also find use in pharmaceutical applications fashioned as inhalers.

Examples of end-use applications other than packaging that benefit from antimicrobial functionality and wherein microbial growth is reduced in the particular end-use of the consumer are components of food processing equipment, such as conveyer belts and their components, components of machines for food cutting and slicing; telephone and cellular phone surfaces; shoe liners and inserts; foam paddings such as mat and rug backings and upholstery components; personal hygiene garments such as diapers, incontinence pads, sanitary napkins, sports pads, tampons and their applicators; medical devices and implants, such as catheters, stents, guide wires, and prostheses; health care materials such as bandages, medical drapes, medical gowns, surgical gloves, gauze strips and pads, syringe holders, IV tubing and bags; and shower curtains and shower curtain liners. In order to impart antimicrobial functionality to the products listed, the product can be treated according to the method of the invention before it is formed or after or at any time during manufacture of the product. For example, in making an antimicrobial shower curtain, material having a surface that is at least 50% by area polyolefin homopolymer or polyolefin copolymer can be treated according to the method of the invention, followed by fashioning a shower curtain from the treated material. Alternatively, the chitosan treatment may be performed after the material is made into a shower curtain. It is believed that the antimicrobial properties of the material will not change significantly.

Any of the above described chitosan treated articles, metal salt treated-chitosan treated articles, or the carboxyl-containing polymer treated articles, may benefit from a further chitosan solution treatment. Included within the scope of this invention are articles that, having received a first treatment with chitosan by the process of the present invention, are further subjected to one or more treatments with metal salt, carboxyl-containing polymer and/or additional chitosan in any order to yield multilayer articles.

The process and articles of the present invention do not employ cross linking agents. The phrase “crosslinking agent” connotes the commonly used di- or tri-functional crosslinking agents. The carboxyl-containing polymers, e.g. polyacrylic acids, are not construed to be crosslinking agents in the context of the present invention.

### EXAMPLES

The present invention is further defined in the following Examples, in which all parts and percentages are by weight and degrees are Celsius. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usage and conditions.

### Materials and Methods

The chitosan used in this study was material commercially available under the registered trademark Chitoclear® from Primex corporation of Norway. The material was used as purchased.

The degree of N-deacetylation of the chitosan sample was ascertained by proton and carbon 13 NMR spectroscopy to be over 85%. The molecular weight of this sample was approximately 74,000.

Treated articles were tested for antimicrobial properties by the Shake Flask Test for Antimicrobial Testing of Materials using the following procedure:

1. Inoculate a single, isolated colony from a bacterial or yeast agar plate culture in 15-25 ml of Trypticase Soy Broth (TSB) in a sterile flask. Incubate at 25-37° C. (use optimal growth temperature for specific microbe) for 16-24 h with or without shaking (select appropriate aeration of specific strain). For filamentous fungi, prepare sporulating cultures on agar plates.

2. Dilute the overnight bacterial or yeast culture into sterile phosphate buffer (see below) at pH 6.0 to 7.0 to obtain approximately 10⁵ colony forming units per ml (cfu/ml). The total volume of phosphate buffer needed will be 50 ml×number of test flasks (including controls). For filamentous fungi, prepare spore suspensions at 10⁵ spores/ml. Spore suspensions are prepared by gently resuspending spores from an agar plate culture that has been flooded with sterile saline or phosphate buffer. To obtain initial inoculum counts, plate final dilutions (prepared in phosphate buffer) of 10⁻⁴ and 10⁻³ onto Typticase Soy Agar (TSA) plates in duplicate. Incubate plates at 25-37° C. overnight.

3. Transfer 50 ml of inoculated phosphate buffer into each sterile test flask containing 0.5 g of material to be tested. Also, prepare control flasks of inoculated phosphate buffer and uninoculated phosphate buffer with no test materials.

4. Place all flasks on a wrist-action shaker and incubate with vigorous shaking at room temperature. Sample all flasks periodically and plate appropriate dilutions onto TSA plates. Incubate at 25-37° C. for 16-48 h and count colonies.

5. Report colony counts as the number of Colony Forming Units per ml (cfu/ml).

6. The Δt value may be calculated as follows: Δt=C-B, where Δt is the activity constant for contact time t, C is the mean log₁₀ density of microbes in flasks of untreated control materials after X hours of incubation, and B is the mean log₁₀ density of microbes in flasks of treated materials after X hours of incubation. Δt is typically calculated at 4, 6, or 24 hours and may be expressed as Δt_{X}.

### Stock Phosphate Buffer

| | |
|---|---|
| Monobasic Potassium Phosphate: | 22.4 g |
| Dibasic Potassium Phosphate: | 56.0 g |
| Deionized Water: | Bring up volume to 1000 ml |

Adjust the pH of the phosphate buffer to pH 6.0 to 7.0 with either NaOH of HCl, filter, sterilize, and store at 4° C. until use. The working phosphate buffer is prepared by diluting 1 ml of stock phosphate buffer in 800 ml of sterile deionized water.

### Example 1

### Oxidation and Grafting of Chitosan onto Polyethylene Tips

Low density polyethylene tips were oxidized with chromic acid according to the literature procedure of J. R. Rasmussen et al. cited supra.

Low density polyethylene tips were extracted with hot 2-propanol in a Soxhlet to clean the outer surface. These tips were then dried under vacuum and treated with a solution of chromium (VI) oxide-water-sulfuric acid (29:42:29 wt. ratio) for 5 to 10 min at 72° C., washed three times with deionized water, and then soaked in conc. nitric acid at 50° C. for 15 min. It was then extensively washed with deionized water, and then soaked in freshly prepared 2% chitosan solution (Chitoclear® solution of Primex, Norway) in 1.5% aqueous acetic acid for 60 min. The tips were then extensively washed with deionized water and dried at 80° C. for 16 h.

Table I shows the antimicrobial effect as determined by the Shake Flask Test method of chitosan grafted to polyethylene tips for the Gram positive bacterium Staphylococcus aureus ATCC 6538, the Gram negative bacteria Escherichia coli ATCC 25922, Pseudomonas aeruginosa ATCC 27853, and Klebsiella pneumoniae ATCC 4352, and the yeast Candida albicans ATCC 10231. The antimicrobial activity is expressed as t at 1 hour and 4 hours of contact time between the microorganisms and the chitosan-treated polyethylene tips. The t is the log reduction of viable cells as calculated between the difference of the log (cfu/ml) of the untreated polyethylene control and the antimicrobial treated polymer.

TABLE 1

| Microorganism | t₁ₕ | t₄ₕ |
|---|---|---|
| Staphylococcus aureus | 2.53 | 5.45 |
| ATCC 6538 | | |
| Escherichia coil ATCC | 5.40 | 5.40 |
| 25922 | | |
| Pseudomonas | 4.71 | 4.26 |
| aeruginosa ATCC 27853 | | |
| Klebsiella pneumoniae | 2.35 | 5.24 |
| ATCC 4352 | | |
| Candida albicans ATCC | 2.67 | 5.20 |
| 10231 | | |

### Example 2

### Oxidation and Grafting of Chitosan onto EVA Bottle Cap Liners

Bottle cap liners made of ethylene vinyl acetate (EVA) was treated as in Example 1. Antimicrobial activity of the treated liners and untreated cap liners was determined by the Shake Flask Test method for the Gram positive bacterium Staphylococcus aureus ATCC 6538, the Gram negative bacteria Escherichia coli ATCC 25922, Pseudomonas aeruginosa ATCC 27853, and Klebsiella pneumoniae ATCC 4352, and the yeast Candida albicans ATCC 10231. After three hours, the treated cap liners exhibited a three-log reduction in viable cells, while the untreated cap liners exhibited no measurable reduction.

### Example 3

### Oxidation and Grafting of Chitosan onto Polypropylene Diaper Liner

A nonwoven polypropylene liner was removed from a commercially available disposable diaper and treated as in Example 1. Antimicrobial activity of the treated diaper liner and an untreated control was determined by the Shake Flask Test method for the Gram positive bacterium Staphylococcus aureus ATCC 6538, the Gram negative bacteria Escherichia coli ATCC 25922, Pseudomonas aeruginosa ATCC 27853, and Klebsiella pneumoniae ATCC 4352, and the yeast Candida albicans ATCC 10231. After three hours, the treated diaper liner exhibited a three-log reduction in viable cells, while the untreated control exhibited no measurable reduction.

### Example 4

### Oxidation and Grafting of Chitosan onto Urethral Stents

A 6 French urethral stent, made of ethylene vinyl acetate, was treated as in Example 1. The treated stent and an untreated stent as a control were packed in Tyvek® pouches and sterilzed with ethylene oxide gas. Antimicrobial activity of the treated stent and an untreated stent was then determined by the Shake Flask Test method for the Gram positive bacterium Staphylococcus aureus ATCC 6538, the Gram negative bacteria Escherichia coli ATCC 25922, Pseudomonas aeruginosa ATCC 27853, and Klebsiella pneumoniae ATCC 4352, and the yeast Candida albicans ATCC 10231. After three hours, the treated stent exhibited a three-log reduction in viable cells, while the untreated stent exhibiteds no measurable reduction.

What is claimed is:

1. An antimicrobial polyolefin article having chitosan grafted thereon.

2. The antimicrobial polyolefin article of claim 1 further comprising one or more compounds selected from the group consisting of metal salts, carboxyl-containing polymers, and combinations thereof.

3. The antimicrobial polyolefin article of claim 1 or 2 wherein the polyolefin is an olefin homopolymer.

4. The antimicrobial polyolefin article of claim 3 wherein the olefin homopolymer is polypropylene or polyethylene.

5. The antimicrobial polyolefin article of claim 4 wherein the olefin homopolymer is selected from the group consisting of low density polyethylene (LDPE), linear low density polyethylene (LLDPE), ultralow density polyethylene (ULDPE), metallocene polyethylene (MePE), high density polyethylene (HDPE) and ultrahigh molecular weight polyethylene (UHMWPE).

6. The antimicrobial polyolefin article of claim 1 or 2 wherein the polyolefin is a copolymer of an ethylene and a vinyl ester.

7. The antimicrobial polyolefin article of claim 6 wherein the vinyl ester is vinyl acetate.

8. The antimicrobial polyolefin article of claim 1 or 2 wherein the polyolefin is a copolymer of an ethylene and an unsaturated acid or ester of the acid selected from the group consisting of acrylic acid, methacrylic acid, 1-8 carbon alkyl acrylates, 1-8 carbon alkyl methacrylate, and mixtures thereof.

9. The antimicrobial polyolefin article of claim 1 or 2 wherein the polyolefin is ethylene/acrylic acid or ethylene/methacrylic acid.

10. The antimicrobial polyolefin article of claim 2 wherein the metal salt is selected from the group consisting of zinc sulfate, copper sulfate, silver nitrate, soluble zinc, copper, and silver salt.

11. The antimicrobial polyolefin article of claim 2 wherein the carboxyl-containing polymer is polyacrylic acid.

12. The antimicrobial polyolefin article of claim 1 or 2 in the form of a container, dropper, tip, container cap, food or beverage dispensing system, applicator, nipple, cosmetics package, or pacifier.

13. A process for preparing antimicrobial polyolefin articles, said process comprising the sequential steps of:
a) providing a polyolefin article,
b) optionally, cleaning the surface of the polyolefin article,
c) contacting the polyolefin article with a solution comprising chromic acid, sulfuric acid or a combination thereof,
d) contacting the polyolefin article of step c) with a solution comprising chitosan,
e) isolating the polyolefin article of step d); and
f) optionally, drying the polyolefin article of step e).

14. The process according to claim 13 wherein the solution comprising chitosan of step d) is an aqueous acetic acid solution.

15. The process according to claim 14, wherein the aqueous acetic acid solution is 0.5% to 5% by volume.

16. The process according to claim 13 wherein the solution comprising chitosan of step d) comprises 0.5% to 1.0% by volume of aqueous acetic acid and 1% to 3% by volume of chitosan.

17. The process according to claim 13 wherein the contacting of step d) with a solution comprising chitosan is performed for 5 to 30 minutes.

18. A method for providing antimicrobial functionality to a polyolefin homopolymer or copolymer film, fabric, or foam surface comprising treating said film, fabric, or foam surface according to the process of claim 13.

19. A method for providing antimicrobial functionality to a beverage or food dispensing system, the method comprising providing a beverage or food dispensing system having a surface of at least 50% by area polyolefin homopolymer or copolymer, and treating said surface according to the process of claim 13.

20. A method for providing antimicrobial functionality to a package, the method comprising providing a package comprising at least one packaging component having a surface of at least 50% by area polyolefin homopolymer or polyolefin copolymer, the packaging component being selected from the group consisting of a liner, lid, cap, film, tray and container, and treating said packaging component according to the process of claim 13.

21. The method of claim 20 wherein said packaging component is formed by extrusion, coextrusion, thermoforming, injection molding, lamination, or blow molding.

22. The method of claim 20 further comprising introducing a beverage or a food into said package.

23. The method of claim 20 wherein the package is a can, box, bottle, jar, bag, or closed-ended tube.

24. The method of claim 20 further comprising introducing a cosmetic, a personal hygiene material, a healthcare material or a combination thereof into said package, wherein said cosmetic, said personal hygiene material or said healthcare material is lipstick, chapstick, eye shadow, eyeliner, mascara, dusting powder, bath powder, blusher, foundation, shampoo, conditioner, deodorant or antiperspirant.

25. The method of claim 20 further comprising introducing a lotion, cream, powder, liquid, solution, suspension, capsule or pill into said package.

26. The method of claim 25 wherein the liquid, solution, or suspension is intended to be applied in the form of discrete drops or spray of droplets.

27. The method of claim 20 wherein said package is an inhaler.

28. A method for providing antimicrobial functionality to an article intended for oral contact, the method comprising providing an article intended for oral contact having a surface that is at least 50% by area polyolefin homopolymer or polyolefin copolymer, wherein said article is a baby bottle nipple, pacifier, orthodontic appliance or component thereof, cup, drinking glass, toothbrush, or teething toy, and treating said article according to the process of claim 13, wherein said article is subsequently contacted to the mouth of a person.

29. A method for providing antimicrobial functionality to an applicator, the method comprising providing an applicator having a surface that is at least 50% by area polyolefin homopolymer or polyolefin copolymer, wherein said applicator is a mascara wand, cosmetics brush, dropper tip, eyeliner applicator, or eye shadow applicator, and treating said applicator according to the process of claim 13.

30. The method of claim 29, further comprising contacting said applicator with a substance to be applied to a surface; and applying said substance with said applicator.

31. A method for providing antimicrobial functionality to a tampon applicator, the method comprising providing a tampon applicator having a surface that is at least 50% by area polyolefin homopolymer or polyolefin copolymer; and treating said tampon applicator according to the process of claim 13.

32. A method for providing antimicrobial functionality on a personal hygiene garment comprising a body-side liner, the method comprising providing a personal hygiene garment comprising a body-side liner having a surface that is at least 50% by area polyolefin homopolymer or polyolefin copolymer and treating said body-side liner according to the process of claim 13.

33. The method of claim 32 wherein the personal hygiene garment is a diaper, incontinence garment, or sanitary napkin.

34. The method of claim 32 wherein the body-side liner comprises a nonwoven polypropylene fabric.

35. A method for providing antimicrobial functionality to food processing equipment, wherein said food processing equipment is a conveyor belt assembly or component thereof, a temporary or permanent food preparation surface, or an element of a machine for cutting food, the method comprising providing said food processing equipment having a surface that is at least 50% by area polyolefin homopolymer or polyolefin copolymer and treating said surface according to the process of claim 13.

36. A method for providing antimicrobial functionality to a shower curtain, the method comprising treating a material having a surface that is at least 50% by area polyolefin homopolymer or polyolefin copolymer according to the process of claim 13 and manufacturing a shower curtain from said treated material.

37. A method for providing antimicrobial functionality to a shower curtain comprising providing a shower curtain having a surface that is at least 50% by area polyolefin homopolymer or polyolefin copolymer, and treating said surface according to the process of claim 13.

38. A method for providing antimicrobial functionality to a telephone or cellular phone, the method comprising providing a telephone or cellular phone having a surface that is at least 50% by area polyolefin omopolymer or polyolefin copolymer and treating said surface according to the process of claim 13.

39. A method for providing antimicrobial functionality to a shoe liner or shoe insert, the method comprising providing a shoe liner or shoe insert having a surface that is at least 50% by area polyolefin homopolymer or polyolefin copolymer and treating said surface according to the process of claim 13.

40. A method for providing antimicrobial functionality to foam padding, the method comprising providing a foam padding having a surface that is at least 50% by area polyolefin homopolymer or polyolefin copolymer and treating said surface according to the process of claim 13.

41. The method of claim 40 wherein said padding is a mat or rug backing or an upholstery component.

42. A method for providing antimicrobial functionality to the surface of a medical device or implant, the method comprising providing a medical device or implant having a surface that is at least 50% by area polyolefin homopolymer or polyolefin copolymer and treating said surface according to the process of claim 13.

43. The method of claim 42 wherein said medical device or implant is a catheter.

44. A method for providing antimicrobial functionality on the surface of a health care material, the method comprising providing a health care material having a surface that is at least 50% by area polyolefin homopolymer or polyolefin copolymer and treating said surface according to the process of claim 13.

45. The method of claim 44 wherein said health care material is a bandage, gauze strip, or gauze pad; medical or surgical drape, gown, head covering, mask, or glove; syringe holder, IV tubing or IV bag.

46. A method for reducing microbial growth in a beverage or food dispensing system, the method comprising manufacturing a beverage or food dispensing system comprising the polyolefin article of claim 1 or 2 and introducing a beverage or food into the beverage or food dispensing system.

47. A method for reducing microbial growth in a package, the method comprising manufacturing a package or packaging component comprising the polyolefin article of claim 1 or 2 and introducing a beverage or a food into said package.

48. The method of claim 20 wherein the package is a can, box, bottle, jar, bag, or closed-ended tube and the packaging component is selected from the group consisting of a liner, lid, cap, film, tray and container.

49. The method of claim 48 further comprising introducing a cosmetic, a personal hygiene material, a healthcare material or a combination thereof into said package.

50. The method of claim 48 further comprising introducing a lotion, cream, powder, liquid, solution, suspension, capsule or pill into said package.

51. The method of claim 48 wherein said package is an inhaler.

52. A method for reducing microbial growth in an article intended for oral contact, the method comprising manufacturing said article which comprises the polyolefin article of claim 1 or 2 and contacting said article to the mouth of a person.

53. The method of claim 52 wherein said article is a baby bottle nipple, pacifier, orthodontic appliance or component thereof, cup, drinking glass, toothbrush, or teething toy.

54. A method for reducing microbial growth in an applicator, the method comprising manufacturing an applicator comprising the polyolefin article of claim 1 or 2, contacting said applicator with a substance to be applied to a surface, and applying said substance to said surface.

55. The method of claim 54 wherein said applicator is a mascara wand, cosmetics brush, dropper tip, eyeliner applicator, or eye shadow applicator.

56. A method for reducing microbial growth of a tampon applicator, the method comprising manufacturing a tampon applicator comprising the polyolefin article of claim 1 or 2 and using said tampon applicator for its intended purpose.

57. A method for reducing microbial growth of a personal hygiene garment, the method comprising manufacturing a personal hygiene garment comprising a body-side liner comprising the polyolefin article of claim 1 or 2, and using the personal hygience garment for its intended purpose.

58. The method of claim 57 wherein the personal hygiene garment is a diaper, incontinence garment, sanitary napkin or sports pad.

59. The method of claim 57 wherein the body-side liner comprises a nonwoven polypropylene fabric.

60. A method for reducing microbial growth on food processing equipment, wherein said food processing equipment is a conveyor belt assembly or component thereof, a temporary or permanent food preparation surface, or an element of a machine for cutting food, the method comprising manufacturing said food processing equipment such that the surface of the food processing equipment comprises the polyolefin article of claim 1 or 2 and using the equipment in its intended manner to process food.

61. A method for reducing microbial growth on a shower curtain comprising manufacturing a shower curtain comprising the polyolefin article of claim 1 or 2 and hanging the shower in the shower for its intended use.

62. A method for reducing microbial growth on a telephone or cellular phone, the method comprising manufacturing a telephone or cellular phone having a surface comprising the polyolefin article of claim 1 or 2 and using the telephone or cellular phone for its intended purpose.

63. A method for reducing microbial growth in a shoe, the method comprising inserting a shoe liner or shoe insert comprising the polyolefin article of claim 1 or 2 into the shoe and wearing the shoe.

64. A method for reducing cell growth in foam padding, the method comprising manufacturing foam padding comprising the polyolefin article of claim 1 or 2 and using said padding as a mat or rug backing or an upholstery component.

65. A method for reducing microbial growth on a medical device or implant, the method comprising manufacturing a medical device or implant comprising the polyolefin of claim 1 or 2 and using the medical device or implant as intended in its medical application.

66. The method of claim 65 wherein said medical device or implant is a catheter.

67. A method for reducing microbial growth on a health care material, the method comprising manufacturing a health care material having a surface comprising the polyolefin article of claim 1 or 2 and contacting the health care material to a patient in its intended use.

68. The method of claim 67 wherein said health care material is a bandage, gauze strip, or gauze pad; medical or surgical drape, gown, head covering, mask, or glove; syringe holder, IV tubing or IV bag.
